(19) United States
(12) Patent Application Publication
(10) Pub. No.: US 2005/0074809 A1
(43) Pub. Date: Apr. 7, 2005

(54) System and method for systematic prediction of ligand/receptor activity

(76) Inventor: Vladimir Brusic, Reservoir (AU)

Correspondence Address: OSTROLENK FABER GERB & SOFFEN, 1180 AVENUE OF THE AMERICAS, NEW YORK, NY, 100368403, US

(21) Appl. No.: 10/471,270

(22) PCT Filed: Mar. 10, 2001
(86) PCT No.: PCT/SG01/00049

(51) Int. Cl. G01N 33/53; G06G 7/48; G06G 7/58; G06F 19/00; G01N 33/48; G01N 33/50

(52) U.S. Cl. 435/7.1; 702/19; 703/11

(57) ABSTRACT

Disclosed is a general system and method, for prediction of binding of peptide-like ligands (peptides) to peptide-like receptors (receptors). Specifically this invention uses non-linear prediction models (including, but not limited to, artificial neural networks), sequence data form ligands and their respective receptors, and known ligand-receptor binding affinities. The representation of ligand-receptor interaction used along with the binding affinity of said interaction is used to train a determining means in a form of a predictive model. Prediction of binding affinity of a novel (not used for training of a predictive model) ligand-receptor interaction, involving a peptide and a particular receptor, involves the combining of representations of both peptide and receptor and presenting that representation to a previously trained predictive model. The system and method can be used as a single predictive model for determination of ligand binding to an individual receptor, or to a group of related receptors. This system and method was validated using data on peptide binding to major histocompatibility complex molecules (MHC) and artificial neural networks (ANN).

### FIELD OF INVENTION

The present invention relates to a system and a method for the systematic identification and prediction of ligand-receptor activity. In particular it relates to the prediction of such activity in peptide and peptide-like ligands in order to identify biologically active compounds and ligands to families of related receptors.

### BACKGROUND

Ligand-receptor interactions are crucial for initiation and regulation of biological responses. A receptor protein resides inside or on the surface of a cell. A receptor has a binding site, which has high activity for a particular signalling molecule. The signalling molecule (i.e. a molecule that binds to a receptor binding-site) is commonly referred to as a ligand. The binding of a ligand molecule initiates a cascade of reactions that induce a change of the state of the affected cell, ultimately resulting in a biological response. A schematic representation of a ligand-receptor induced response is given in FIG. 1. Examples of ligands include hormones, pheromones, neurotransmitters, peptides, drugs, and small molecules, among others. An effector transduces the signal generated by a ligand-receptor binding (also termed recognition). The signal transduction induces a reaction (or a series of events), such as transport of ions or molecules that ultimately induce the new state of the affected cell. The reaction can be amplified by a secondary signal or can self-amplify. This new state of the cell results in a biological response, such as enzyme activation or deactivation, protein synthesis, protein stabilisation, and release of hormones or transmitters, among others.

Understanding the ligand-receptor interaction is important for the analysis of biological responses, and related applications. One receptor may bind multiple ligands, or the same ligand may be recognised by multiple receptors. One cell may have multiple receptors of the same type. Different cells may have the receptors of the same type. These receptors sometimes belong to families that have large number of variants. Screening a family of receptors for their ligands requires exhaustive experimentation and is not feasible, because of the excessive experimental cost. Thus a significant effort has been invested in developing computational methods for modelling of the ligand-receptor interactions. The approaches to modelling the ligand-receptor interactions include molecular modelling, statistical methods, and various heuristics.

Peptides that bind major histocompatibility complex (MHC), particularly those that are naturally processed, are potential vaccine candidates for immunisation against cancer, infectious disease or autoimmune disease. MHC molecules represent the receptor families in which there are several types of receptors (i.e. class I and class II). One MHC receptor can recognise multiple ligands, one ligand may bind several receptor variants, one cell typically has multiple variants of MHC receptor of the same type, and one cell may have multiple types of MHC receptors. Although the number of peptides that can bind to a specific human leukocyte antigen (HLA or human MHC) molecule is large, it is two to three orders of magnitude smaller than the number of peptides that can be generated by the degradation of protein antigens. Short peptides displayed on the surface of cells, in conjunction with MHC molecules that are recognised by T-cells are termed T-cell epitopes.

T-cell epitope mapping, including HLA-peptide-binding studies, is currently one of the most intensively researched areas of molecular and cellular immunology. Because of the extensive HLA allelic variation (more than a 1000 HLA allelic variants have been determined to date) a systematic laboratory approach to T-cell epitope mapping, even of a single protein antigen, is impractical for the reasons outlined above. Computational prediction of peptide-MHC binding is thus a useful methodology for efficient and practical pre-selection of potential T-cell epitopes.

A MHC molecule has a binding groove that accommodates binding peptide. The binding groove contains binding pockets, which provide for most of binding interactions with the side chains of anchoring amino acids of a binding peptide. There are six binding pockets in MHC class I molecules, and the same number in MHC class II binding molecules. Most peptides binding to MHC class I are 8-10 amino acids long, while MHC class II peptides are 10-30 amino acids long with a 9-mer long binding core. MHC molecules are highly polymorphic, with most of polymorphism contained within the amino acids that form binding groove and its pockets. One analysis of the peptide binding environment to HLA molecules may be provided by defining the amino acids of HLA molecules that are in the proximity of linear positions of amino acids within a binding peptide.

A major use of biologically active compounds is in the discovery and design of medicinal drugs. Computational screening methods have been used for identification and engineering of biologically active compounds, and for data mining from molecular databases. The advances in genomics and proteomics have facilitated a shift from traditional methods of direct antimicrobial screening towards rational drug design (Rosamond J. and Allsop A. (2000), “Harnessing the power of the genome in the search for new antibiotics” Science 287, 1973-1976). Methods such as phage display libraries facilitate experimental screening (Hoogenboom H. R., Griffiths A. D., Johnson K. S., Chiswell D. J., Hudson P. and Winter G. (1991), “Multi-subunit proteins on the surface of filamentous phage: methodologies for displaying antibody (Fab) heavy and light chains”, Nucleic Acids Research 19, 4133-4137) of protein-ligand interactions and have been used for screening of antibody libraries, protein-receptor, and protein-ligand interactions. Computational screening methods have proven powerful in searching for compounds that interact with a known molecular structure such as receptor or an enzyme. The tools for identification of biologically active compounds from combinatorial libraries using three-dimensional computational simulations (virtual screening) have been developed and extensively used (Makino S., Ewing T. J., and Kuntz I. D. (1999), “DREAM++: flexible docking program for virtual combinatorial libraries”, Journal of Computer Aided Molecular Design 13, 513-532). The advantage of viral screening methods is in highly increased efficiency relative to the experimental methods, however these virtual screening methods are relatively complicated and slow for large-scale screening. They are often combined with statistical methods for improving the speed and accuracy (Broughton H. B. (2000), “A method for including protein flexibility in protein-ligand docking: improving tools for database mining and virtual screening”, Journal of Molecular Graphics and Modelling 18, 247-257). Statistical methods that provide high speed and high accuracy are desirable for further improvement of the efficiency of discovery of drug targets.

Several computational models for prediction of MHC-binding peptides have been developed. These models use methods based on binding motifs (see for example Rammensee H., Bachmann J., Emmerich N. P., Bachor O. A. and Stevanovic S. (1999), “SYFPEITHI: database for MHC ligands and peptide motifs”, Immunogenetics 50, 213-219, and WO 93/20103, WO 94/11738, WO 97/34621, WO 96/03140 and WO 97/41440), quantitative matrices (see for example Mallios R. R. (1999), “Class II MHC quantitative binding motifs derived from a large molecular database with a versatile iterative stepwise discriminant analysis meta-algorithm”, Bioinformatics 15, 432439, and U.S. Pat. No. 6,037,135), artificial neural networks—ANN (Brusic V., Rudy G. and Harrison L. C. (1994), “Prediction of MHC binding peptides using artificial neural networks”, in Stonier R. J. and Yu X. S., (eds), Complex Systems: Mechanism of Adaptation, pp. 253-260, IOS Press, Amsterdam/OHMSHA Tokyo and U.S. Pat. No. 5,933,819), hidden Markov models—HMM (Mamitsuka, H. (1998), “Predicting peptides that bind to MHC molecules using supervised learning of hidden Markov models”, Proteins 33, 460-74) and molecular modelling (Lim J. S., Kim S., Lee H. G., Lee K. Y., Kwon T. J. and Kim K. (1996), “Selection of peptides that bind to the HLA-A2.1 molecule by molecular modeling”, Molecular Immunology 33, 221-230) for example. Some of these methods have been successfully applied in practice for identification of novel T-cell epitopes (see for example WO 98/32456). Each of these methods require a definition of a distinct model (motif, matrix, ANN, HMM, molecular model) for prediction of peptide binding to a given MHC allele. For large-scale screening, there is a need for prediction of peptides binding across multiple MHC molecules. Multiple quantitative matrices for identification of promiscuous HLA class II ligands have been reported (Sturniolo T., Bono E., Ding J., Raddrizzani L., Tuereci O., Sahin U., Braxenthaler M., Gallazzi F., Protti M. P., Sinigaglia F. and Hammer J. (1999), “Generation of tissue-specific and promiscuous HLA ligand databases using DNA microarrays and virtual HLA class II matrices”, Nature Biotechnology 17, 555-561). However, a method that utilises a single model that can predict peptide binding to a multiplicity of MHC alleles is still lacking.

Molecular modelling uses detailed knowledge of the crystal structure of MHC molecules and of protein-peptide interactions. Molecular modelling had been proven useful for visualization and detailed analysis of pocket interactions in clefts of various MHC molecules (Zhang, C., Anderson, A. & DeLisi, C. (1998), “Structural principles that govern the peptide-binding motifs of class I MHC molecules”, J. Mol. Biol. 281, 929-947 (3D modelling. Date: 4 Sep. 1998)), but being computationally intensive, this methodology is currently less useful for large-scale screening of potential MHC binding peptides. Computational threading algorithm uses the co-ordinates of solved complexes to evaluate the interactions of peptide amino acids with MHC contact residues, and results in a peptide score that reflects its binding energy (Altuvia Y., Sette A., Sydney J., Southwood S., Margalit H. (1997), “A structure-based algorithm to predict potential binding peptides to MHC molecules with hydrophobic binding pockets”, Hum. Immunol. 58, 1-11 (Computational threading. November 1997)). The use of computational threading for prediction of peptide binding to a range of MHC class I molecules has also been reported (Schueler-Furman O., Elber R, Margalit H. (1998), “Knowledge-based structure prediction of MHC class I bound peptides: a study of 23 complexes”, Fold. Des. 549-564 (3D modelling. Date: 1998)). A variety of other computational methods for prediction of peptides and their properties have been reported. These methods include 3D modelling techniques, such as docking, thermodynamics simulations, use of topological indices, periodicity algorithms, or secondary structure prediction, among others.

An artificial neural network (ANN) is an information-processing system consisting of the densely interconnected structure of computational elements. An ANN consists of many self-adjusting processing elements co-operating in a densely interconnected network. ANN architecture (FIG. 2) comprises a great number of simple, summing processors that work in parallel and transmit signals between each other via modifiable weighted links. The activation pattern of units depends of the strength of input signals, connection weights, and unit activation thresholds. Input to this ANN is a representation of peptide sequences; the output signal representing peptide binding activity. Training an ANN involves adjusting the connection weights and activation thresholds until it learns binding patterns, gaining the ability to match peptides (input) with their binding affinities (output). A neural network of the appropriate size can universally approximate any smooth function to any desired degree of accuracy. However it is relatively difficult to elucidate generalised rules that characterise training data from a trained neural network.

The advantages of ANNs, of a particular relevance for dealing with biological data, are: a) ANNs are adaptive and self-refine with the addition of new data, b) they can handle imperfect data and tolerate data containing errors, c) they are suited to deal with non-linear problems, and d) after being initially defined, ANN models are easy to use and refine.

Peptides are usually represented as character strings where each character represents an amino acid. To convert character strings into a format appropriate as input to an ANN three distinct representations of the input data have been investigated earlier (see Table 3, Example 3 below). “Rep 20” assigns a unique binary string to each of the 20 possible amino acids, but does not encode any of the physical properties (features) which characterise them. “Rep 6” assigns a 6-place string where each place is a scalar value for a feature (hydrophobicity, volume, charge, aromatic side chain, hydrogen bonds) or a correction bit. “Rep 9” is an intermediate representation using a feature-based grouping of amino acids. The following amino acid features are encoded: hydrophobicity, positive charge, negative charge, aromatic side chain, aliphatic side chain, small size, bulky size; two correction bits are added for distinguishing similar amino acids. Each peptide is represented as a continuous string of digits, the length depending on the representation. Peptide binding is usually represented as a numerical value with low numbers representing non-binding.

In comparison to methods based on binding motifs, quantitative binding matrices, multiple binding matrices and modelling techniques, ANN and HMM predictions are based on more sophisticated computational algorithms that allow capturing of the complex patterns that define peptide binding. An ANN- or a HMM-based predictive model is trained using a set of peptides and their binding affinities to a particular receptor; For prediction of peptide binding to a query peptide (of unknown binding affinity to a given receptor) data will be presented to the trained ANN or HMM model which outputs the prediction value. ANNs have been reported to have superior accuracy compared to predictions of MHC-binding peptides compared to methods that use binding motifs or binding matrices (Brusic V., Rudy G., Honeyman M., Hammer J. and Harrison L. C. (1998a), “Prediction of MHC class II-binding peptides using an evolutionary algorithm and artificial neural network”, Bioinformatics 14, 121-130; Borras-Cuesta F, Golvano J, Garcia-Granero M, Sarobe P, Riezu-Boj J, Huarte E, Lasarte J. (2000), “Specific and general HLA-DR binding motifs: comparison of algorithms”, Human Immunology, 61, 266-278). Comparison of the performances of quantitative matrices and ANNs for prediction of peptide binding to the class I molecule HLA-A*0201 has indicated that quantitative matrices have high specificity whereas ANNs have high sensitivity of predictions (Gulukota K., Sidney J., Sette A. and Delisi C. (1997), “Two complementary methods for predicting peptides binding major histocompatibility complex molecules”, Journal of Molecular Biology 267, 1258-1267), with higher accuracy again observed for HMM-based predictions of MHC-binding peptides (Mamitsuka, H. (1998), “Predicting peptides that bind to MHC molecules using supervised learning of hidden Markov models”, Proteins 33, 460-74).

It is an object of the present invention to provide a method which can identify and predict ligand I receptor activity, in particular activity of peptide and peptide-like ligands and to provide a system for implementing such a method.

It is another object of the present invention to provide a method which can be used to predict of the activity of molecules for which no experimental data is available, but which may be refined by inclusion of new experimental data.

It is a further object of the present invention to provide a method which enables large-scale screening of molecules and which is genaralisable for prediction of ligand-receptor interactions for various receptor families, in particular to enable identification of peptide-like ligands to families of related receptors.

It is also an object of the present invention to provide a method that utilises a single model that can predict peptide binding to a multiplicity of MHC alleles.

### BRIEF DESCRIPTION OF THE INVENTION

Accordingly, the present invention provides a method for predicting interaction of ligands and receptors, comprising the steps of:
a) representing ligand-receptor interaction by combining representations of ligand interaction sites and representations of receptor interaction sites;
b) training a determining means with representations characterising said at least one ligand-receptor interaction of known or estimated affinity; and
c) using the trained determining means to analyse representations of at least one ligand-receptor interaction of unknown affinity.

Additionally, the present invention provides, in a computer based system for predicting interaction'of ligands and receptors:
1) means for representing ligand-receptor interaction using a combination of representations of ligand interaction sites and representations of receptor interaction sites;
2) means for training a computer or other determining means with representations characterising said at least one ligand-receptor interaction of known or estimated affinity; and
3) means for analysing representations of at least one ligand-receptor interaction of unknown affinity.

The method and system of the present invention differ from existing methods and systems in that they combine both representations of ligand and receptor for each single data training point and are thus based on the characteristics of the ligand-receptor interactions rather than on the characteristics of either ligand or receptor component in isolation. As a result the present invention facilitates the use of a single model for prediction of binding activity of a ligand to multiple receptors.

In another aspect the invention relates to a computer program, residing on a computer-readable medium, for identifying relative affinity of ligand-receptor interactions, comprising instructions for causing a computer to:
a) represent a ligand-receptor interaction by combining representations of a receptor interaction site and representations of a ligand receptor site;
b) train a computer or other determining means with representations characterising at least one ligand-receptor interaction of known or estimated affinity;
c) apply to the computer or other determining means representations of at least one test ligand-receptor interaction of unknown affinity, using the same representation form as used in training the computer or other determining means; and
d) analyse each applied test ligand-receptor interaction in order to predict the affinity of each test ligand-receptor interaction.

The present invention is particularly useful in predicting the interaction between ligands and receptors where the ligand is a peptide and the receptor is a peptide receptor. Preferably the input data comprises representations of interactions of peptides binding MHC molecules (of class I or class II) or HLA molecules.

Preferably, the determining means is selected from the group consisting of an ANN, a HMM, a multiple regression means and a Bayesian network.

In essence the present invention allows the use of a single model to predict binding affinity and biological activity on the basis of a characterisation of the reciprocal relationship between ligand and receptor. The reciprocal relationship between ligand and receptor is characterised in terms of parameters which relate to the interaction of the two components, rather than parameters which describe the components in isolation from each other. It is thus the characteristics of the interaction or binding event which become important rather that the characteristics of the individual components themselves. In this way the behaviour of multiple ligands towards a single receptor, or a single ligand to multiple receptors, m ay be assessed.

### DESCRIPTION OF THE DRAWINGS

The present invention is described in greater detail below with reference to the accompanying drawings in which:

FIG. 1 is a schematic representation of a ligand-receptor interaction;

FIG. 2 is a schematic representation of an ANN showing layers of units which transmit signals through connecting arcs;

FIG. 3 is a high-level representation of a general prediction system of peptide-ligand interaction;

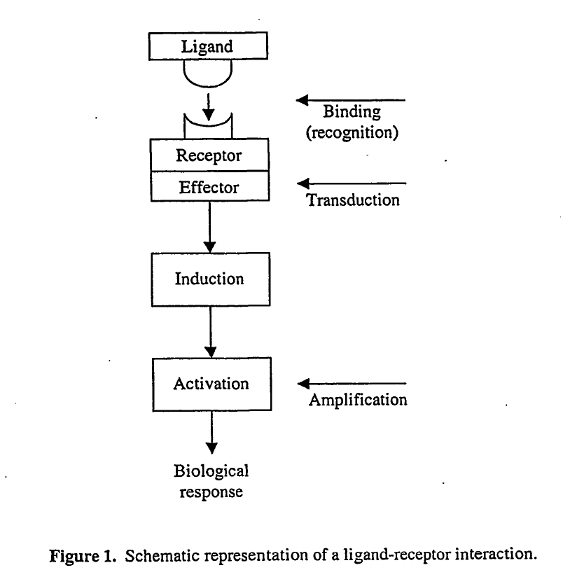

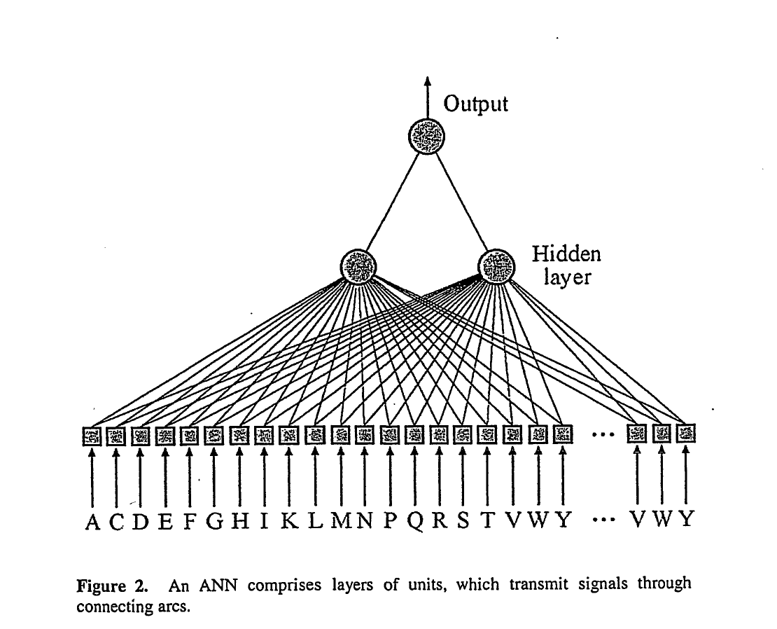

FIG. 4 illustrates the process of building a representation of the ligand-receptor interaction for use in the present invention; the contact amino acids are those that are involved in interaction, or that provide for structural integrity of the interaction.
A) Identification of amino acid in both ligand and receptor that are involved in the ligand-receptor interaction. This step can use information derived by using various methods, such as crystallography, molecular modelling, homology modelling, functional studies, mutation binding studies, etc.
B) Identification of contact amino acids locations in linear sequences.
C) Removal of non-contact amino acids from the sequence and fragment merging.
D) Combining representations of contact residues for the definition of a specific ligand-receptor interaction.

FIG. 5 shows the identification of peptide contact residues within the HLA-A*0201 molecule and representation of the contact site;

FIG. 6 shows a representation of the binding interaction site between the MAGE-3 peptide FLWGPRALV and its receptor HLA-A*0201;

FIG. 7 shows the identification of peptide contact residues within the HLA-DRB1*0101 chain of the HLA-DR1(0101) molecule and representation of the peptide contact site;

FIG. 8 shows the identification of peptide contact residues within the HLA-DRA1*0101 chain of the HLA-DR1(0101) molecule and representation of the peptide contact site;

FIG. 9 shows a representation of the binding interaction site between the invariant chain peptide PKPPKPVSKMRMATPLLQALPMG and its receptor HLA-DR1(0101);

FIGS. 10-12 show representations of the receptor interaction site in beta chains of eight HLA-DR molecules;

FIG. 13 shows representative peptides, corresponding HLA-DR molecules, peptide binding affinity and the representation of the interaction;

FIG. 14 shows a conversion of a representative interaction comprising a peptide core FAGKNTDLE that does not bind HLA-DR1(0101);

FIG. 15 shows the receptor interaction sites in beta chains of the 17 studied HLA-DR molecules, and HLA-DRB1*0801 (HLA-DRB1*0801 was used for model training, but not for predictions); and

FIG. 16 shows the short representation of the beta chain contact sites for the 17 studied HLA-DR molecules and HLA-DRB1*0801 (HLA-DRB1*0801 was used for model training, but not for predictions).

### DETAILED DESCRIPTION

The present invention is based on the use of an ANN, a HMM or some other suitable determining means for the prediction of ligand-receptor binding activity (for example identification of peptide binding to MHC molecules) building a single model which can predict ligand binding to a multiplicity of different receptors with high accuracy. It facilitates cyclical refinement of predictive models for improved accuracy by inclusion of new experimental data. In addition it facilitates high accuracy predictions of peptide binding to MHC molecules for which no experimental data are available. It also enables large-scale screening of MHC-binding peptides and has the advantage that it is genaralisable for prediction of peptide-receptor interactions for various receptor families. The method of the present invention is genaralisable for prediction of peptide-receptor interactions for various interactions involving, but not limited to, MHC molecules, T-cell receptors, immunoglobulins, ion channel blockers and protein cleavage.

Building and application of a statistical (i.e. regression-based, ANN, HMM, etc.) prediction system for ligand-receptor interactions typically involves several stages:
a) Representation of known ligand-receptor interactions in a format useful for training a determining means;
b) Training the determining means;
c) Representing an unknown (or test) ligand-receptor interaction in the same format as defined in step a);
d) Predicting the affinity of the unknown ligand-receptor interaction.

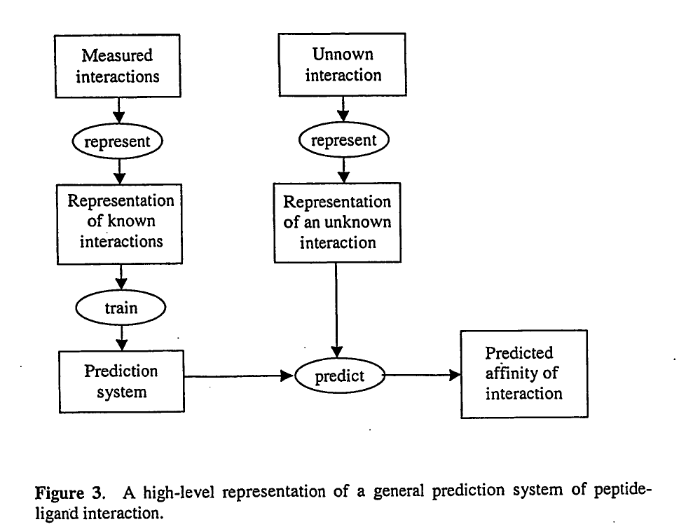

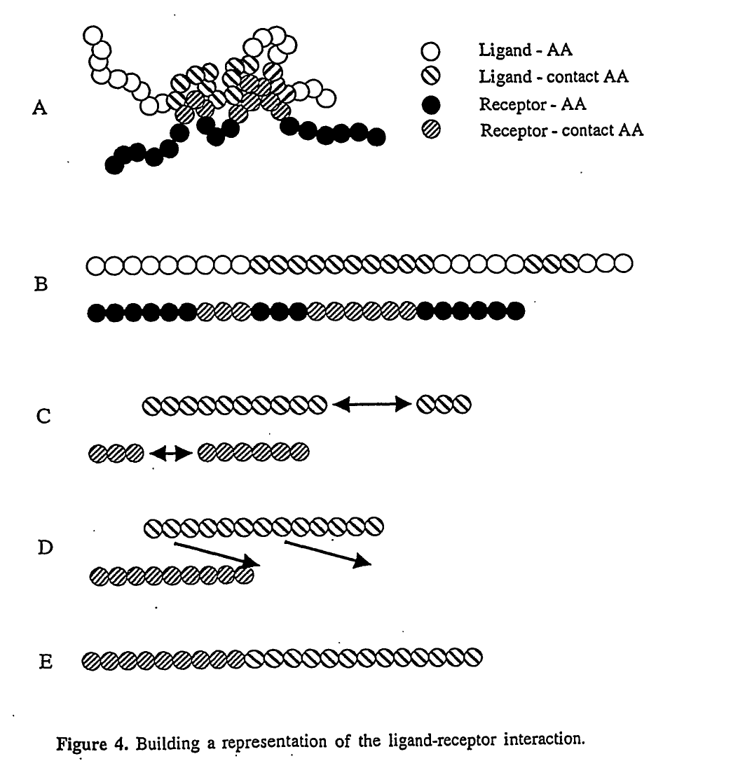

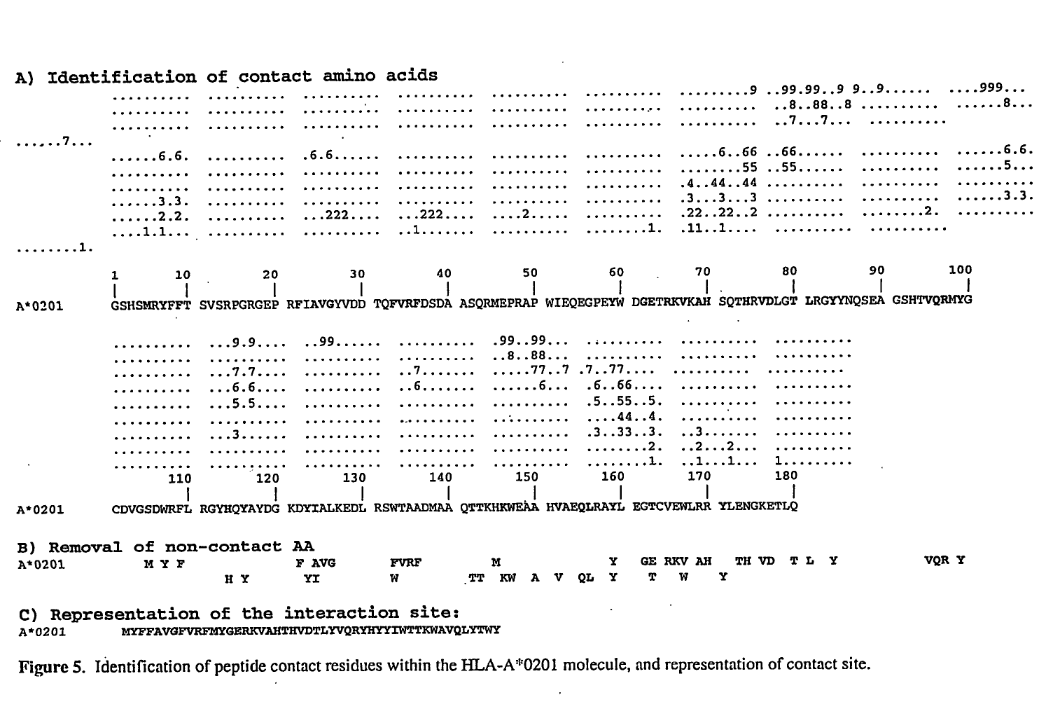

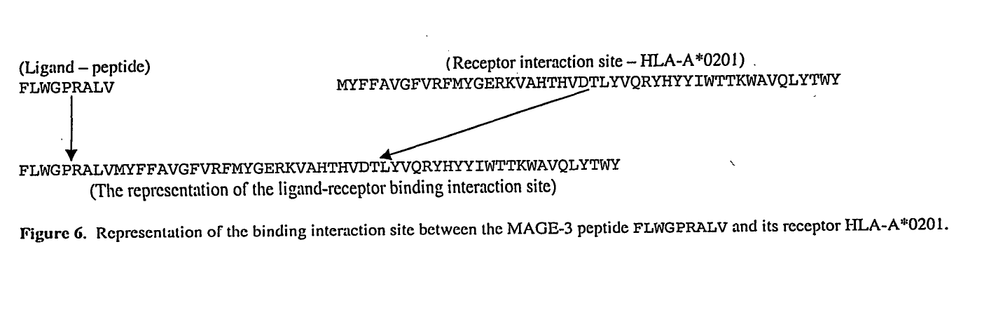

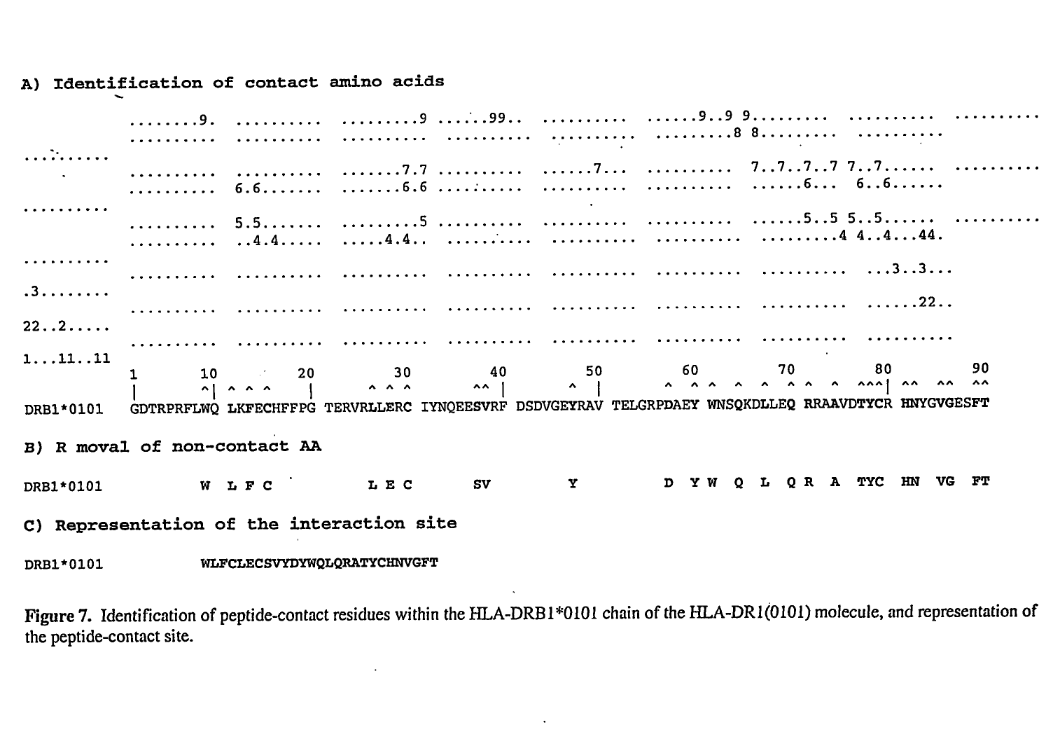

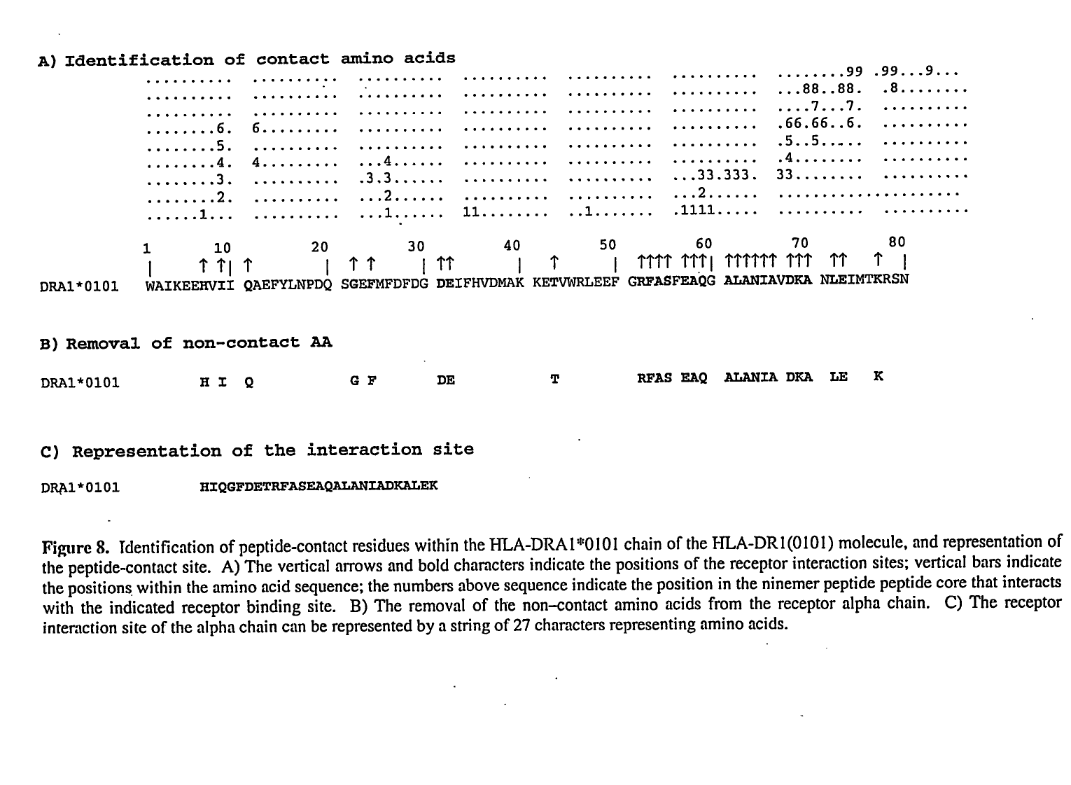

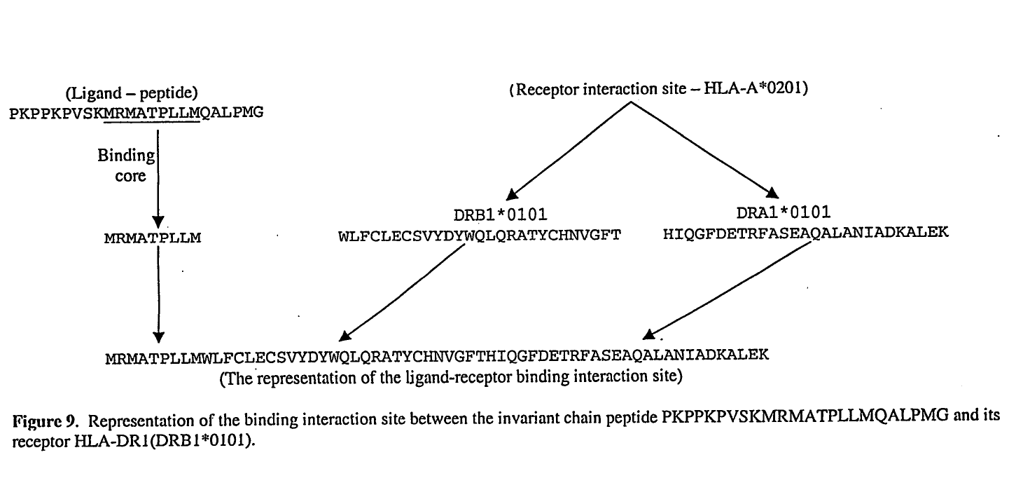

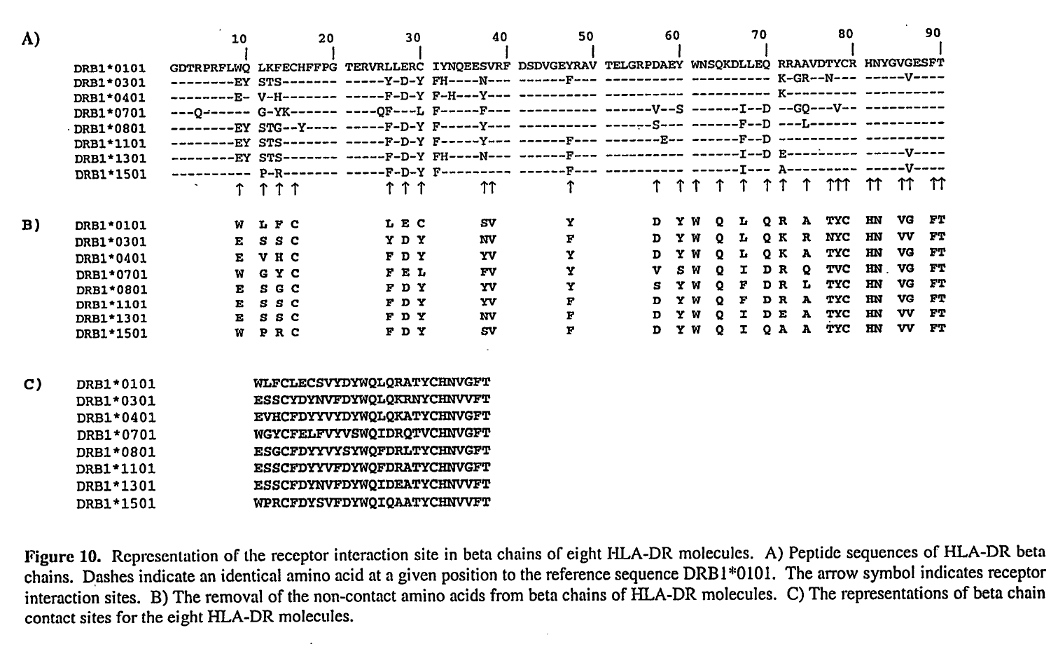

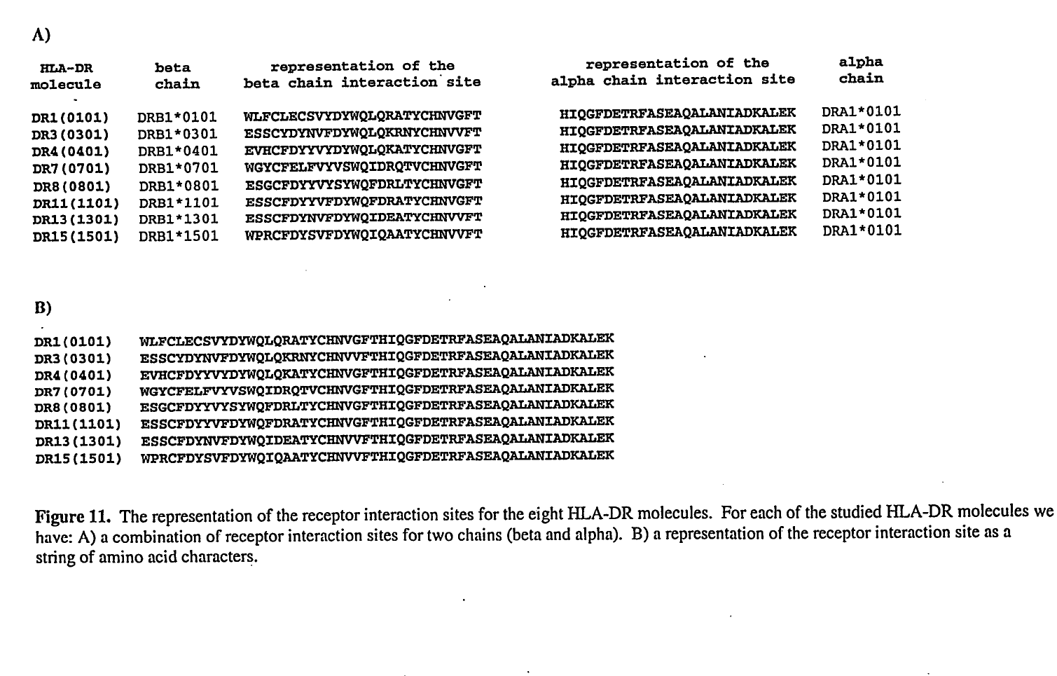

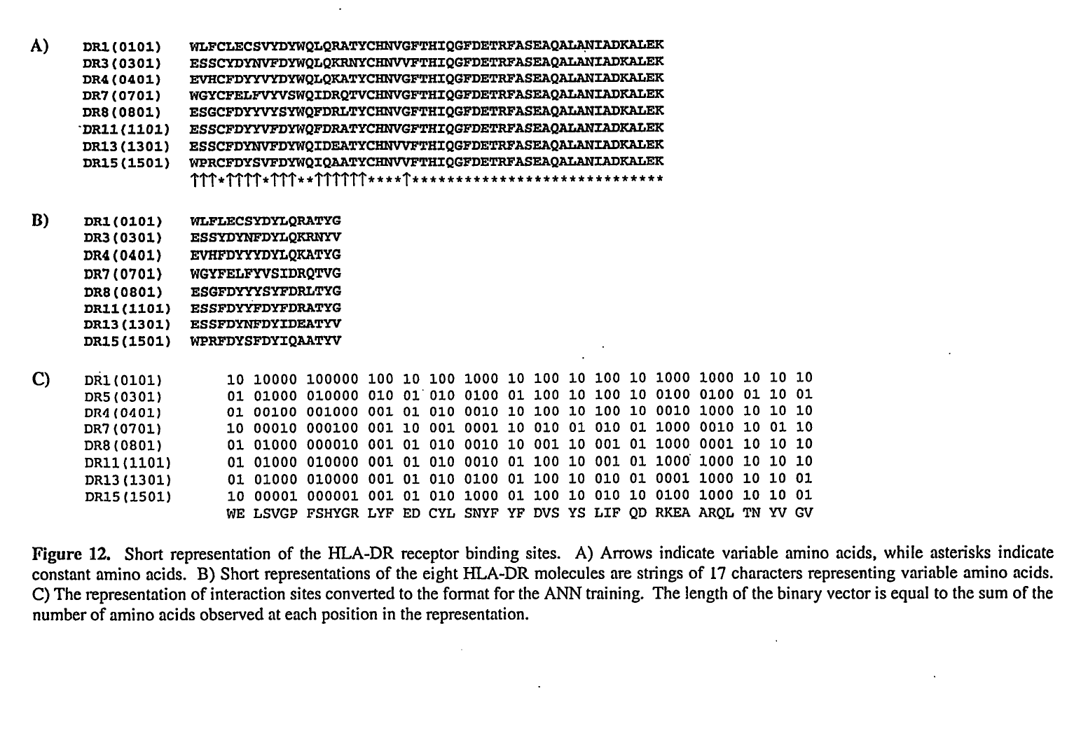

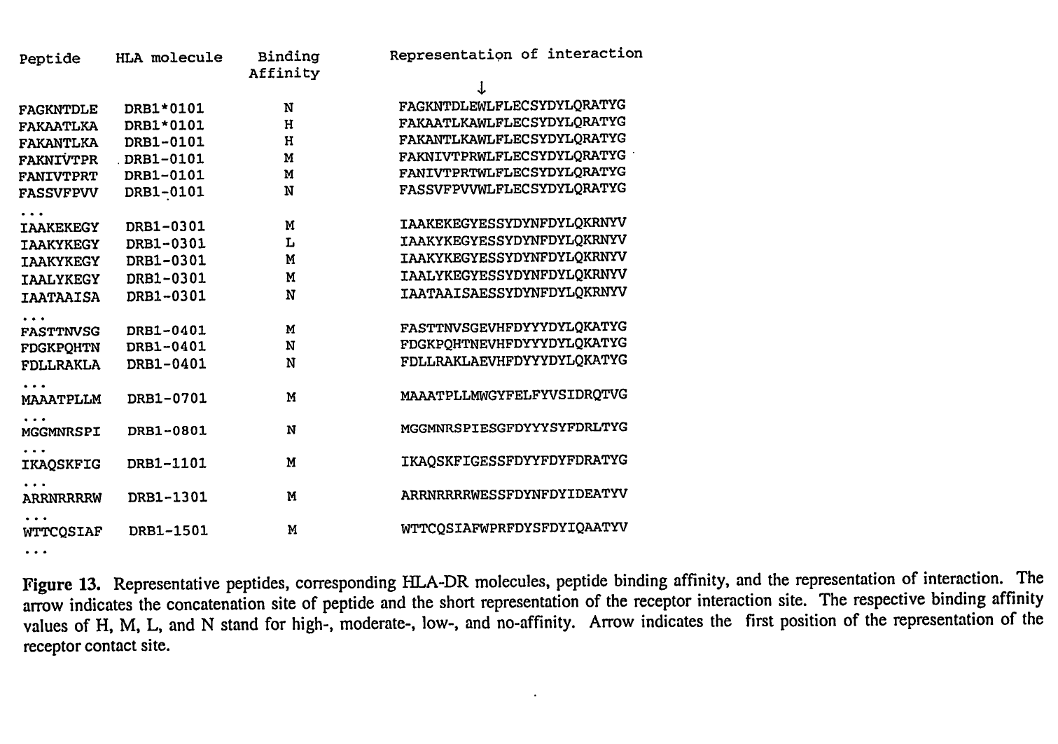

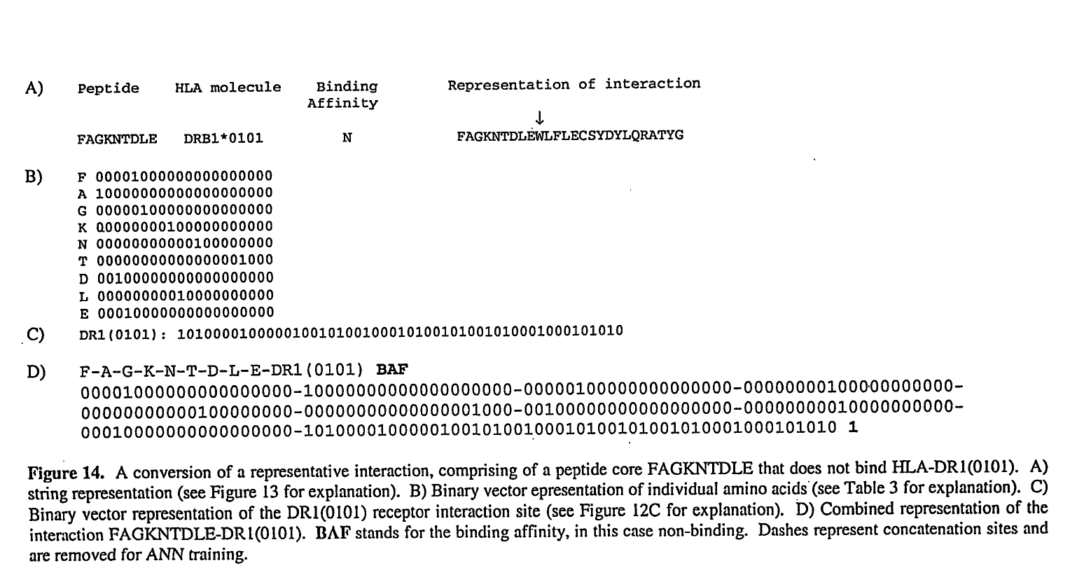

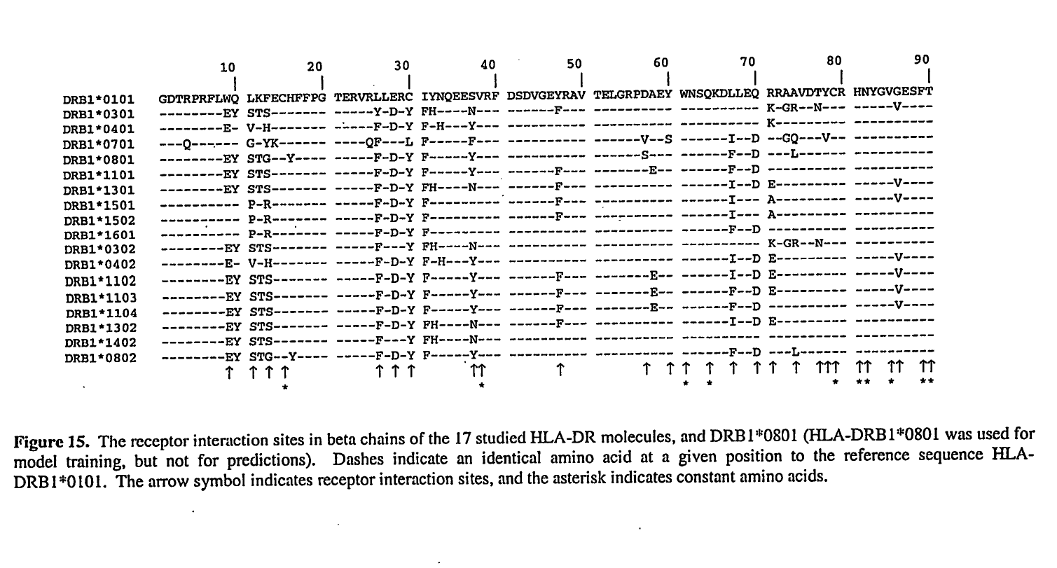

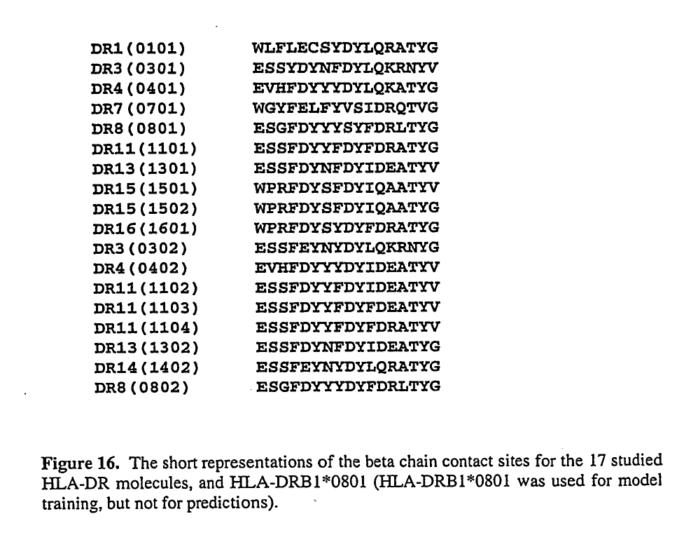

A schematic high-level representation of a prediction system for ligand-receptor activity is shown in FIG. 3. The critical part of the method and system of this invention is the description and the representation of the ligand-receptor interactions by combining ligand contact sites and receptor contact sites uniformly for a family of related receptors. Taking the example of a peptide-receptor interaction the steps of the present invention include, but are not limited to, the following:
a) Identification of the contact elements in a receptor sequence (receptor contact sites) from a representative known structure. The contact elements are amino acids that directly or indirectly affect a ligand-receptor interaction.
b) Identification of the contact elements in a ligand sequence (ligand contact sites) from a representative known structure. The contact elements are amino acids that directly or indirectly affect a ligand-receptor interaction.
c) Align ligand sequences from known ligand-receptor interaction of the studied family.
d) Align receptor sequences from known ligand-receptor interaction of the studied family.
e) Remove non-contact amino acids identified in steps a) and b).
f) Combine ligand and receptor contact sites into the interaction representation for each known ligand-receptor interaction.
g) Remove invariant sites from the alignment (optional).
h) Represent ligand-receptor interaction in a format suitable for use with a determining means.
i) Train the determining means.
j) Represent a ligand-receptor interaction of unknown affinity in the format suitable for use with the determining means (following the procedure described in steps a) to h)).
k) Predict the affinity of the unknown ligand-receptor interaction.

The invention involves the production and use of a novel data representation that combines experimental and structural information, the representation of the ligand-receptor interaction being defined by combination of representation of interaction sites of both the ligand and the receptor. The invention uses a degenerate representation of ligand and receptor interaction sites thus allowing the use of minimal representation. Using a non-linear statistical technique such as for example an ANN or a H1) enables training of a single model for prediction of ligand binding to multiple related receptors.

A computer-based general system and method for prediction of binding of peptides or peptide-like ligands (ligands) to peptide-like receptors (receptors) operates as follows. Identification of contact sites within the ligand and representing ligand interaction sites by combining ligand contact sites (in an arbitrary order) and similar identification of contact sites within the receptor and representing receptor interaction sites by combining receptor contact sites (in an arbitrary order) facilitates representation of the ligand-receptor interaction by combining representations of receptor interaction site and ligand receptor site (in an arbitrary order). Using such ligand-receptor representations a determining means (such as an ANN, a HMM or a multiple regression system) is trained with input data characterising instances of ligand-receptor interactions of known binding affinity. After training, test data representing a test ligand-receptor interaction of unknown affinity (using the same representation form as for training the determining means) is applied and analysed to predict the affinity of the test ligand-receptor interaction. The method of present invention may be set out in the form of a computer program, residing on a computer-readable medium and may be implemented using a computer programmed with one of the above mentioned determining means.

Therefore, in a computer-based general system and method for prediction of binding of peptide ligands to peptide receptors, the steps may be summarised as follows:
1) Identification of contact sites within the ligand and representing ligand interaction sites by combining ligand contact sites (in an arbitrary order);
2) Identification of contact sites within the receptor and representing receptor interaction sites by combining receptor contact sites (in an arbitrary order);
3) Representing ligand-receptor interaction by combining representations of receptor interaction site and ligand receptor site (in an arbitrary order);
4) Training a detemming means with input data characterising instances of known ligand-receptor interactions, using said representations and known affinity of each interaction;
5) Applying to the determining means test data representing at least one test ligand-receptor interaction of unknown affinity using the same representation form as for training the determining means; and
6) Analysis of each applied test ligand-receptor interaction to produce a prediction of affinity of ligand-receptor interaction, and computing such predictions.

As indicated above the present invention uses a non-linear statistical technique, which may be selected from the group consisting of an artificial neural network, a hidden Markov model, multiple regression and a Bayesian network. The use of such a technique facilitates cyclical refinement of predictive models for improved accuracy by inclusion of new experimental data as it becomes available. Where no experimental data with which to train the determining means exists the training process may be based on estimated binding affinity produced using other methods. For example, if binding activity of a ligand-receptor interaction is unknown, but there is experimental evidence of biological activity, a reasonable estimate of binding affinity can be deduced and used for training a predictive model.

The system and method according the present invention is generally applicable to data sets based on any type of ligand-receptor interaction. Typically the training data may comprise representations of interactions of peptides binding major histocompatibility complex (MHC) molecules (class I or class II) or peptides binding human leukocyte antigen (HLA) molecules.

The operation of the invention is illustrated by the following non-limiting Examples.

### EXAMPLES

Coding Procedure

The coding procedure for a peptide binding to a single receptor includes several steps (FIG. 4):
a) Identification of amino acid residues involved in interaction-FIG. 4A
b) Linear representation of amino acids involved in interaction-FIG. 4B
c) Removal of non-contact residues to form representation fragments-FIG. 4C
d) Merging fragments to form the representation of interaction-FIG. 4D
e) Optional removal of amino acids that are constant (identical in all receptor variants).
f) Selection of amino acid representation and conversion into format suitable for training a prediction system

### Example 1

Peptide PLWGPRALV of the mage-3 antigen (SWISSPROT:MAG3_HUMAN) binds HLA-A*0201 molecule (Kawakami Y. and Rosenberg S.A. (1996) “T-cell recognition of self peptides as tumour rejection antigens”, Immunologic Research 15, 179-190). The interaction site of the peptide with the cleft of the HLA-A*0201 molecule is the whole length of the peptide. The positional binding environments of peptides have been resolved by crystallography (Bjorkman P. J., Saper M. A., Samraoui B., Bennett W. S., Strominger J. L., Wiley D. C. (1987), “Structure of the human class I histocompatibility antigen, HLA-A2”, Nature 329, 506-512). The HLA-A peptide residue positional environments are summarised in Table 1. The process of obtaining the representation of the interaction of the said peptide and said receptor is shown in FIG. 5. HLA-A*0201 has 48 contact amino acids on the surface of the binding groove that constitute peptide interaction site (FIG. 5A). Removing non-contact amino acids (FIG. 5B) and concatenation of contact amino acids results in the representation of the interaction site of the HLA-A*0201 receptor (FIG. 5C). Putting together the peptide interaction site, in this example whole 9-mer peptide, and the receptor interaction site, results in the representation of the interaction site (FIG. 6).

TABLE 1
Peptide residue positional environments for HLA
class I molecules. Adapted from Chelvanayagam
G. (1996) “A roadmap for HLA-A, HLA-B,
and HLA-C peptide binding specificities”,
Immunogenetics 45, 15-26.

| Position | Contact residues |
|---|---|
| P1 | 5, 7, 33, 59, 62, 63, 66, 99, 159, 163, 167, 171 |
| P2 | 7, 9, 24, 25, 26, 34, 35, 36, 45, 62, 63, 66, 67, 70, 99, 159, |
| | 163, 167 |
| P3 | 7, 9, 62, 66, 70, 97, 99, 114, 152, 155, 156, 159, 163 |
| P4 | 62, 65, 66, 69, 70, 155, 156, 159 |
| P5 | 69, 70, 73, 74, 97, 114, 116, 152, 155, 156, 159 |
| P6 | 7, 9, 22, 24, 66, 69, 70, 73, 74, 97, 99, 114, 116, 133, 147, |
| | 152, 155, 156 |
| P7 | 73, 77, 97, 114, 116, 133, 146, 147, 150, 152, 155, 156 |
| P8 | 73, 76, 77, 80, 97, 143, 146, 147 |
| P9 | 70, 73, 74, 76, 77, 80, 81, 84, 95, 96, 97, 114, 116, 123, 124, |
| | 142, 143, 146, 147 |
| All | 5, 7, 9, 22, 24, 25, 26, 33, 34, 35, 36, 45, 59, 62, 63, 65, 66, 67, |
| | 69, 70, 73, 74, 76, 77, 80, 81, 84, 95, 96, 97, 99, 114, 116, 123, |
| | 124, 133, 142, 143, 146, 147, 150, 152, 155, 156, 159, 163, |
| | 167, 171 |

### Example 2

Peptide PKPPKPVSKMRMATPLLMQALPMG of class II invariant chain (SwissProt Acc. P04233) binds HLA-DRB1*0101 molecule (Chicz R. M., Urban R. G., Gorga J. C., Vignali D. A., Lane W. S. and Strominger J L. (1993) “Specificity and promiscuity among naturally processed peptides bound to HLA-DR alleles”, Journal of Experimental Medicine 178, 27-47). The interaction site of the peptide with the cleft of the HLA-DR1(DRB1*0101) molecule is the 9-mer binding core (MATPLLM).

The positional binding environments of peptides have been resolved by crystallography (Stern L. J., Brown J. H., Jardetzky T. S., Gorga J. C., Urban R. G., Strominger J. L., Wiley D. C. (1994), “Crystal structure of the human class II MHC protein HLA-DR1 complexed with an influenza virus peptide”, Nature 368, 215-221). The HLA-DR peptide residue positional environments are summarised in Table 2. The process of obtaining the representation of the said peptide and the said receptor is shown in FIG. 7. HLA-DR1(0101) is a dimer consisting of two chains, HLA-DRB1*0101 and HLA-DRA1*0101. HLA-DR1(0101) has 54 contact amino acids on the surface of the binding groove that constitute peptide interaction site (Table 2). There are 27 contact amino acids in HLA-DRB1*0101 (FIG. 7A) and 27 contact amino acids in HLA-DRA1*0101 (FIG. 8A). Removing non-contact amino acids (FIGS. 7B and 8B) and concatenation of the contact amino acids results in the representation of the interaction site of the HLA-DR1(0101) receptor, namely interaction sites in the beta chain HLA-DRB1*0101 (FIG. 7C) and in the alpha chain HLA-DRA1*0101 (FIG. 8C). Putting together the peptide interaction site, in this example the 9-mer peptide binding core, and the receptor interaction sites, see results in the representation of the interaction site (FIG. 9).

TABLE 2
Peptide residue positional environments for
HLA-DRB1*0101 molecule. Adapted from Chelvanayagam
G. (1997), “A roadmap for HLA-DR peptide binding
specificities”, Human Immunology 58, 61-69.

| Peptide | Contact residues | |
|---|---|---|
| Position | Alpha chain | Beta chain |
| P1 | 7, 24, 31, 32, 43, 52, | 81, 82, 85, 86, 89, 90 |
| | 53, 54, 55 | |
| P2 | 9, 24, 54 | 77, 78, 81, 82, 85 |
| P3 | 9, 22, 24, 54, 55, 57, | 74, 78, 82 |
| | 58, 59, 61, 62 | |
| P4 | 9, 11, 24, 62 | 13, 15, 26, 28, 70, 71, 74, 78, 79 |
| P5 | 9, 62, 65 | 11, 13, 30, 67, 70, 71, 74 |
| P6 | 9, 11, 62, 63, 65, 66, 69 | 11, 13, 28, 30, 67, 71, 74 |
| P7 | 65, 69 | 28, 30, 47, 61, 64, 67, 70, 71, 74 |
| P8 | 64, 65, 68, 69, 72 | 60, 61 |
| P9 | 69, 70, 72, 73, 76 | 9, 30, 37, 38, 57, 60, 61 |
| All | 7, 9, 11, 22, 24, 31, 32, | 9, 11, 13, 15, 26, 28, 30, 37, 38, 47, |
| | 43, 52, 53, | 57, 60, 61, 64, 67, 70, 71, 74, 77, |
| | 54, 55, 57, 58, 59, 61, | 78, 79, 81, 82, 85, 86, 89, 90 |
| | 62, 63, 64, 65, 66, | |
| | 68, 69, 70, | |
| | 72, 73, 76 | |

Data Preparation for ANN Training

Receptor families, such as MHC molecules, typically share a common conserved structure. The specificity of the ligand-receptor interaction is thus defined by amino acids at fixed positions across the aligned linear sequences of the receptors. The representations of known peptide/MHC interactions can therefore be used to train an ANN for prediction of peptide binding to a range of related receptors. The interaction sites can be determined within the multiple aligned sequences of receptors as described previously (see Example 2). The representation of an individual interaction can be described as LIS-RIS-BA, where LIS stands for ligand interaction site, RIS for receptor interaction site, and BA for measured strength of the interaction (the binding affinity). The general representation of the training data for interactions between ligands and multiple related receptors can be represented as LISₐ-RIS_{b}-BA_{a, b} where a or b represent known interactions of any specific ligand (a) that binds a specific receptor (b).

Implementation

### Example 3

Binding affinity of a number of peptides have been measured for eight HLA-DR molecules DR1, DR3, DR4, DR7, DR8, DR11, DR13, and DR15 (Table 4). Binding cores of the peptides have been determined by using binding motifs (Rammensee H., Bachmann J., Emmerich N. P., Bachor O. A. and Stevanovic S. (1999), “SYFPEITHI: database for MHC ligands and peptide motifs”, Immunogenetics 50, 213-219) or matrix methods (Brusic V., Zeleznikow J., Sturniolo T., Bono E. and Hammer J. (1999), “Data cleansing for computer models: a case study from immunology”, Proceedings of ICONIP99, The sixth International Conference on Neural Information Processing, IEEE, 603-609). The representation of the receptor interaction sites for the beta chains of eight HLA-DR molecules is given in FIG. 10. The alpha chain is identical for the eight HLA-DR molecules (see FIG. 8). The representation of the receptor interaction sites for the eight HLA-DR molecules is given in FIG. 11. Constant amino acids provide no basis for discrimination between the receptor sites. Short representation of the receptor interaction sites, created by removing amino acids that are constant across the studied HLA-DR molecules, is shown in FIG. 12. Representative peptides and the representation of the interactions are shown in FIG. 13. A specific example of the conversion of a representative interaction to a format that can be used for training an ANN is given in FIG. 14. Other representations are also possible, including those shown in Table 3.

TABLE 3
Amino acids representations. An “a” stands for “10”.
(Adapted from Brusic V., Rudy G. and Harrison L. C. (1994),
“Prediction of MHC binding peptides using artificial neural
networks”, in Stonier R. J. and Yu X. S., (eds), Complex
Systems: Mechanism of Adaptation, pp. 253-260,
IOS Press,Amsterdam/OHMSHA Tokyo.)

| | Representation | | |
|---|---|---|---|
| Amino Acid | “20” | “6” | “9” |
| Alanine (A) | 10000000000000000000 | 53500a | 100001000 |
| Cysteine (C) | 01000000000000000000 | 45500a | 000001001 |
| Aspartate (D) | 00100000000000000000 | a5001a | 001001000 |
| Glutamate (E) | 00010000000000000000 | a6001a | 001000100 |
| Phenylalanine (F) | 00001000000000000000 | 185a0a | 100100101 |
| Glycine (G) | 00000100000000000000 | 525000 | 000001000 |
| Histidine (H) | 00000010000000000000 | 565010 | 010100111 |
| Isoleucine (I) | 00000001000000000000 | 37500a | 100010011 |
| Lysine (K) | 00000000100000000000 | a7a02a | 010000111 |
| Leucine (L) | 00000000010000000000 | 375000 | 100010001 |
| Methionine (M) | 00000000001000000000 | 375005 | 100000100 |
| Asparagine (N) | 00000000000100000000 | 655020 | 000001000 |
| Proline (P) | 00000000000010000000 | 555000 | 100000010 |
| Glutamine (Q) | 00000000000001000000 | 66502a | 000000100 |
| Arginine (R) | 00000000000000100000 | a8a046 | 010000110 |
| Serine (S) | 00000000000000010000 | 645010 | 000001011 |
| Threonine (T) | 00000000000000001000 | 55501a | 000001010 |
| Valine (V) | 00000000000000000100 | 365007 | 100011010 |
| Tryptophan (W) | 00000000000000000010 | 0a5a10 | 100100110 |
| Tyrosine (Y) | 00000000000000000001 | 285a18 | 000100111 |
| Any AA (X) | 11111111111111111111 | 555555 | 111111111 |
| No AA (gap) | 00000000000000000000 | 000000 | 000000000 |

A fully connected three-layer feed forward ANN was trained using the PlaNet software (Miyata, Y. (1991), “A user's guide to Planet Version 5.6.”, Computer Science Department, University of Colorado). The training set consisted of binding and non-binding 9-mer peptides (Table 4). The ANN architecture comprised 231 input units (corresponding to the binary representation of 9-mer peptides) three hidden layer units, and a single output unit. The learning algorithm was error back-propagation (Rummelhart, D., Hinton, G. E., and Williams, R. (1986), “Learning Internal Representations by Error Propagation”, Parallel and Distributed Processing: Exploration in the Microstructure of Cognition, Vol. 1, D. Rummelhart and J. McClelland (Eds.), MIT Press, Cambridge, Mass., 318-362). The ANN training was performed for 300 cycles. The values for momentum and learning rate were 0.5 and 0.2, respectively. The interactions were represented as described in FIG. 14. The output value was scaled 0-10, representing a range from no affinity to the very high binding affinity. Binding scores used for ANN training were 1, 4, 6, and 8 for no-, low-, moderate-, and high-affinity binders, respectively.

Overlapping peptides from the bee venom protein API m1 were experimentally tested for binding to seven HLA-DR alleles, HLA-DR1(0101), DR3(0301), DR4(0401), DR7(0701), DR11(1101), DR13(1301), and DR15(1501) (Texier C, Pouvelle S, Busson M, Herve M, Charron D, Menez A, Maillere B. (2000), “HLA-DR restricted peptide candidates for bee venom immunotherapy” Journal of Immunology, 164, 3177-3184). Binding of API m1 peptides to these molecules was predicted using individual ANNs and the method of the present invention. We trained individual ANNs using data shown in Table 4, as described previously (Brusic V., Rudy G., Honeyman M., Hammer J. and Harrison L. C. (1998), “Prediction of MHC class II-binding peptides using an evolutionary algorithm and artificial neural network” Bioinformatics 14, 121-130). The results of predictions are shown in Table 5. The comparison of the results shows that the predictive power of the present method (Table 5B) is comparable to those of the individual predictions (Table 5A). The apparently low values of the specificity are an artifact of the peptide selection. Because of the length of the overlapping peptides (18 amino acids long, with 5 amino acids overlap), each single false positive prediction of a nine-mer peptide produced two to three false positives. Using the present invention, high affinity binders, which are the best T-cell epitope candidates, were predicted with high)accuracy. Of 21 high-affinity binders, only two peptides or 9.5% (one each for HLA-DR13 and -DR15) were false negatives. Of 38 moderate binders nine peptides or 23.7% were false negatives. Of 34 low binders, 12 peptides or 35.3% were false negatives. Overall, these results for the method of the present invention correspond to the respective values of sensitivity of 91.5%, 76.3%, and 64.7% for high-, moderate-, and low-binders. The present method has the additional advantage that all the predictions were produced using a single predictive model.

TABLE 4
Peptides reported to bind or not to bind to the eight HLA-DR molecules.
Binding affinities have been defined as high, moderate, low, or
non-binding according to the scheme defined in the MHCPEP database
(Brusic V., Rudy G. and Harrison L. C. (1998c), “MHCPEP—a database
of MHC-binding peptides: update 1997”, Nucleic Acids Research, 26,
368-371).

| Binding | HLA-DR allele | | | | | | | |
|---|---|---|---|---|---|---|---|---|
| affinity | 0101 | 0301 | 0401 | 0701 | 0801 | 1101 | 1301 | 1501 |
| High | 113 | 33 | 133 | 25 | 0 | 64 | 0 | 20 |
| Moderate | 130 | 129 | 88 | 138 | 44 | 126 | 58 | 37 |
| Low | 21 | 43 | 83 | 47 | 0 | 43 | 23 | 20 |
| None | 880 | 234 | 448 | 304 | 85 | 191 | 289 | 123 |

Validation

### Example 4

The ANN training data were the same as in Example 3 (Table 4) and comprised measured peptide binding for eight HLA-DR molecules. Trained network was used to predict peptide binding to 17 variants of the HLA-DR molecules. The sequences of the beta chains (amino acids 1 through 90) of the studied HLA-DR molecules are shown in FIG. 15. The representation of the receptor interaction site was determined using the same procedure as described in the Example 3. The short representations of the receptor interaction sites are shown in FIG. 16. The network trained as described in Example 3, using peptide binding data for eight HLA-DR molecules (see Table 4) was used for prediction of peptide binding to the 17 HLA-DR molecules (described in FIGS. 15 and 16).

The hepatitis C virus core protein (HCV core 1b) peptides have been experimentally tested for binding to a number of HLA-DR alleles including HLA-DR1(0101), DR3(0301), DR3(0302), DR4(0401), DR4(0402), DR7(0701), DR8(0802), DR11(1101), DR11(1102), DR11(1103), DR11(1104), DR13(1301), DR13(1302), DR14(1402), DR15(1501), DR15(1502), and DR16(1601) (Borras-CuestaF, Golvano J, Garcia-Granero M, Sarobe P, Riezu-Boj J, Huarte E, Lasarte J. (2000), “Specific and general HLA-DR binding motifs: comparison of algorithms”, Human Immunology, 61, 266-278). Binding of HCV core 1b peptides to these molecules was predicted using the present method. The results of predictions (Table 6) show that the predictive power of the present invention is of a reasonable accuracy, comparable to the results of predictions from Example 3. Using the present invention peptide binding to HLA-DR molecules for which binding data are not available can be predicted. For some variants, such as HLA-DR11(1102), DR11(1103), and DR1(1104), the accuracy of predictions is very similar to the prediction to the base variant HLA-DR11(1101). For some variants, such as HLA-DR3(0302), and DR4(0402), the accuracy of predictions is somewhat lower than that of the base variant. The present invention can therefore be used for prediction of ligand binding to the whole families of related receptors.

TABLE 5
Results of prediction of peptide binding to
eight studied HLA-DR molecules.
5A) Individual ANNs

| Af- | HLA-DR molecule | | | | | | | | | | | | | |
|---|---|---|---|---|---|---|---|---|---|---|---|---|---|---|
| finity | 0101 | | 0301 | | 0401 | | 0701 | | 1101 | | 1301 | | 1501 | |
| | TP | FN | TP | FN | TP | FN | TP | FN | TP | FN | TP | FN | TP | FN |
| HB | 3 | 1 | 4 | 0 | 0 | 1 | 2 | 2 | 0 | 1 | 1 | 1 | 5 | 0 |
| MB | 1 | 4 | 3 | 0 | 6 | 5 | 1 | 4 | 8 | 0 | 1 | 2 | 3 | 0 |
| LB | 1 | 1 | 5 | 2 | 2 | 2 | 4 | 4 | 4 | 0 | 0 | 3 | 3 | 3 |
| | TN | FP | TN | FP | TN | FP | TN | FP | TN | FP | TN | FP | TN | FP |
| NB | 12 | 7 | 12 | 4 | 6 | 8 | 8 | 5 | 9 | 8 | 18 | 3 | 9 | 7 |

| | | | | | | | |
|---|---|---|---|---|---|---|---|
| SE | 0.46 | 0.86 | 0.5 | 0.41 | 0.92 | 0.25 | 0.79 |
| SP | 0.63 | 0.75 | 0.43 | 0.61 | 0.52 | 0.86 | 0.56 |

5B) Present Invention

| Af- | HLA-DR molecule | | | | | | | | | | | | | |
|---|---|---|---|---|---|---|---|---|---|---|---|---|---|---|
| finity | 0101 | | 0301 | | 0401 | | 0701 | | 1101 | | 1301 | | 1501 | |
| | TP | FN | TP | FN | TP | FN | TP | FN | TP | FN | TP | FN | TP | FN |
| HB | 4 | 0 | 4 | 0 | 1 | 0 | 4 | 0 | 1 | 0 | 1 | 1 | 4 | 1 |
| MB | 2 | 3 | 3 | 0 | 10 | 1 | 4 | 1 | 6 | 2 | 2 | 1 | 2 | 1 |
| LB | 1 | 1 | 5 | 2 | 2 | 2 | 4 | 4 | 4 | 0 | 2 | 1 | 4 | 2 |
| | TN | FP | TN | FP | TN | FP | TN | FP | TN | FP | TN | FP | TN | FP |
| NB | 13 | 6 | 11 | 5 | 7 | 7 | 7 | 6 | 12 | 5 | 16 | 6 | 10 | 6 |

| | | | | | | | |
|---|---|---|---|---|---|---|---|
| SE | 0.63 | 0.86 | 0.81 | 0.71 | 0.85 | 0.62 | 0.71 |
| SP | 0.68 | 0.69 | 0.50 | 0.54 | 0.47 | 0.76 | 0.62 |

HB—high affinity binders,
MB—moderate affinity binders,
LB—low affinity binders,
NB—non-binders.
TP—true positives (predicted binders and experimental binders)
TN—true negatives (predicted non-binders, experimental binders),
FP—false positives (predicted binders, experimental non-binders),
FN—false negatives (predicted non-binders, experimental binders).
Sensitivity, the proportion of true binders predicted correctly, was calculated as SE = TP/(TP + FN).
Specificity, the proportion of true non-binders predicted correctly, was calculated by formula SP = TN/(TN + FP).

TABLE 6
Prediction results for 17 studied HLA-DR molecules.

| HLA-DR | TP | FN | TN | FP | SE | SP |
|---|---|---|---|---|---|---|
| 0101 | 5 | 3 | 22 | 5 | 0.63 | 0.81 |
| 1501 | 9 | 4 | 18 | 4 | 0.69 | 0.82 |
| 1502 | 4 | 5 | 21 | 5 | 0.44 | 0.81 |
| 1601 | 3 | 4 | 21 | 7 | 0.43 | 0.75 |
| 0301 | 3 | 1 | 20 | 11 | 0.75 | 0.65 |
| 0302 | 1 | 2 | 22 | 10 | 0.33 | 0.69 |
| 0402 | 4 | 13 | 16 | 2 | 0.24 | 0.89 |
| 0401 | 4 | 4 | 20 | 7 | 0.5 | 0.74 |
| 1101 | 9 | 5 | 14 | 7 | 0.64 | 0.67 |
| 1102 | 6 | 2 | 21 | 6 | 0.75 | 0.78 |
| 1103 | 3 | 4 | 20 | 8 | 0.43 | 0.71 |
| 1104 | 8 | 4 | 18 | 5 | 0.67 | 0.78 |
| 1301 | 3 | 5 | 20 | 7 | 0.38 | 0.74 |
| 1302 | 5 | 7 | 20 | 3 | 0.42 | 0.87 |
| 1402 | 4 | 5 | 21 | 5 | 0.44 | 0.81 |
| 0701 | 3 | 5 | 20 | 7 | 0.38 | 0.74 |
| 0802 | 5 | 8 | 16 | 6 | 0.38 | 0.73 |

The description of symbols is given in the legend of Table 5.

“Comprises/comprising” when used in this specification is taken to specify the presence of stated features, integers, steps or components, but does not preclude the presence or addition of one or more other features integers, steps, components or groups thereof.

1. A method for predicting interaction of ligands and receptors, comprising the steps of:
a) representing ligand-receptor interaction by combining representations of ligand interaction sites and representations of receptor interaction sites;
b) training a determining means with representations characterising said at least one ligand-receptor interaction of known or estimated affinity; and
c) using the trained determining means to analyse representations of at least one ligand-receptor interaction of unknown affinity.

2. A method according to claim 1 wherein the ligand-receptor interaction representations comprise representations of molecules selected from the group consisting of peptides binding class I MHC molecules, peptides binding class II MHC molecules and peptides binding HLA molecules.

3. A method according to claim 1 the ligand interaction sites are represented by identifying contact sites within the ligand and combining said contact sites in an arbitrary order.

4. A method according to claim 1 the receptor interaction sites are represented by identifying contact sites within the receptor and combining said contact sites in an arbitrary order.

5. A method according to claim 1, wherein the determining means is selected from the group consisting of an ANN, a HMM, a multiple regression means and a Bayesian network.

6. A computer based system for predicting interaction of ligands and receptors:
a) means for representing ligand-receptor interaction using a combination of representations of ligand interaction sites and representations of receptor interaction sites;
b) means for training a computer or other determining means with representations characterising said at least one ligand-receptor interaction of known or estimated affinity; and
c) means for analysing representations of at least one ligand-receptor interaction of unknown affinity.

7. A computer based system according to claim 6 wherein the ligand-receptor interaction representations comprise representations of molecules selected from the group consisting of peptides binding class I MHC molecules, peptides binding class II MHC molecules and peptides binding HLA molecules.

8. A computer based system according to claim 6 he ligand interaction sites are represented by identifying contact sites within the ligand and combining said contact sites in an arbitrary order.

9. A computer based system according to claim 6 the receptor interaction sites are represented by identifying contact sites within the receptor and combining said contact sites in an arbitrary order.

10. A computer based system according to claim 1, wherein the determining means is selected from the group consisting of an ANN, a HMM, a multiple regression means and a Bayesian network.

11. A computer program, residing on a computer-readable medium, for identifying relative affinity of ligand-receptor interactions, comprising instructions for causing a computer to:
a) represent a ligand-receptor interaction by combining representations of a receptor interaction site and representations of a ligand receptor site;
b) train a computer or other determining means with representations characterising at least one ligand-receptor interaction of known or estimated affinity;
c) apply to the computer or other determining means representations of at least one test ligand-receptor interaction of unknown affinity, using the same representation form as used in training the computer or other determining means; and
d) analyse each applied test ligand-receptor interaction in order to predict the affinity of each test ligand-receptor interaction.

12. A computer program according to claim 11 wherein the ligand-receptor interaction representations comprise representations of molecules selected from the group consisting of peptides binding class I MHC molecules, peptides binding class II MHC molecules and peptides binding HLA molecules.

13. A computer program according to claim 11 the ligand interaction sites are represented by identifying contact sites within the ligand and combining said contact sites in an arbitrary order.

14. A computer program according to claim 11 the receptor interaction sites are represented by identifying contact sites within the receptor and combining said contact sites in an arbitrary order.

15. A computer program to claim 11, wherein the determining means is selected from the group consisting of an ANN, a HMM, a multiple regression means and a Bayesian network.
